(19) United States
(12) Patent Application Publication
(10) Pub. No.: US 2012/0316403 A1
(43) Pub. Date: Dec. 13, 2012

(54) Consumer Performance Assessment and Monitoring System

(76) Inventor: Josh N. Hogan, Los Altos, CA (US)

(21) Appl. No.: 13/487,182

(22) Filed: Jun. 2, 2012

Related U.S. Application Data
(60) Provisional application No. 61/520,378, filed on Jun. 9, 2011.

(51) Int. Cl. A61B 5/00

(52) U.S. Cl. 600/300

(57) ABSTRACT

A method, apparatus and system for consistent and repeated assessment and monitoring of performance of activities yielding information for predicting probabilities of specific illnesses or degenerative diseases occurring or for the recognition of aptitudes, skills and interests that can lead to improved quality of life and economic well-being by making better informed life choices. The invention includes using a web-site, Internet portal, or consumer device to provide custom activities for a user. At least some of the activities have measurable and quantifiable aspects, which provide a mechanism for analyzing the performance of activities and comparing such measurable and quantifiable aspects with statistical information derived from similar measurements on a large number of a peer group, or measurement previously acquired for the same individual to provide guidance to an individual.

### CROSS REFERENCES TO RELATED APPLICATIONS

The invention described in this US patent application, docket number JH090929US claims priority from provisional patent application, docket number JH090929PR, application No. 61/520,378 filed on Jun. 9, 2011. The invention relates to using a web-site, portal, or consumer device, to repeatedly perform custom activities, selected by a user and a mechanism for analyzing the performance of at least some of the activities to provide an assessment of some aspect of the performance of the user.

### FIELD OF THE INVENTION

The invention relates to using such web-sites, portals, or consumer devices and ensuing assessments for early diagnosis of an illness, or early warning of onset of an illness, or a learning disability, or suitability for a particular career or activity. The invention also relates to the use of consumer devices, such as audio or video devices, to assist in performance assessment and monitoring.

### BACKGROUND OF THE INVENTION

Healthcare costs are a significant barrier to the health and well-being of many individuals who for financial or other reasons are excluded from medical insurance programs. Early diagnosis of potentially negative health issues can enable preventative measures that can mitigate adverse health and economic consequences, however such early diagnosis is often unavailable for cost or other reasons including, but not limited to: lack of coverage by insurance programs; requirement to remain at place of employment; and convenient geographical access.

Wellness, quality of life and economic well-being can also be enhanced by recognition of gradual changes in physical or mental capabilities and access to information on how to best deal with such changes. Furthermore systematic assessment or recognition of aptitudes, skills and interests can also lead to improved quality of life and economic well-being by using information derived from such systematic assessment or recognition to guide in making life choices, such as, education emphasis and employment choices.

Early diagnosis, medical monitoring and professional assessment of aptitudes, skills and interests is typically expensive, time consuming and not systematically available to a large number of individuals. There is therefore an unmet need for a low cost readily assessable and convenient system for such early diagnosis, medical monitoring and professional assessment of aptitudes, skills and interests.

### SUMMARY OF THE INVENTION

The invention taught herein meets at least all of the aforementioned unmet needs. The invention provides a method, apparatus and system for consistent and repeated assessment and monitoring of performance of activities that can yield information useful for predicting the probability of a specific illness or degenerative disease occurring. It also provides information for the systematic assessment or recognition of aptitudes, skills and interests which can lead to improved quality of life and economic well-being by using information derived from such systematic assessment or recognition to guide in making life choices, such as, education emphasis and employment choices.

The invention involves using a web-site, Internet portal, or consumer device such as an iPhone, iPod or cell phone, to provide custom activities for a user. Typical activities include, but are not limited to: puzzles; tests; entertainment; learning; business activities. Specific examples of such activities include, but are not limited to: crossword puzzles, sudoku, hearing and visual acuity tests, memory tests, response time tests, movies, music, books, travel, learning musical instruments, learning foreign languages, performing financial or mathematical analysis.

At least some of the activities have measurable and quantifiable aspects, such as, time to completion, error rate, memory recall, recognition of patterns, spelling accuracy, accuracy of reproduction of sounds, visual and or audio resolution, etc. Such measurable and quantifiable aspects provide a mechanism for analyzing the performance of at least some of the activities. Such measurable and quantifiable aspects are herein referred to as performance parameters.

The availability of audio, video and haptic (or tactile) devices coupled with ubiquitous low cost computer devices enables sophisticated measurement of physical and mental capabilities as well as assessment or recognition of aptitudes, skills and interests.

The widespread access to the Interned enables storing and tracking performance characteristics or parameters. Such stored performance characteristics or parameters can be used to make comparisons, on a statistical bases, with other typical health conditions, behavior or life-choices of individuals with similar performance characteristics or parameters to provide guidance to individuals.

The availability of such devices coupled with Internet access enables an opportunity to provide significant personal or individual wellness or performance programs by repeated assessment and monitoring of physical and mental performance and characteristics.

In order to track performance over time similar or identical tasks may be repeated and current performance parameters compared with previous performance parameters. An example of such tracking of the performance of an activity would be to present the same sudoku puzzle that had been previously played and comparing the current performance with previous performance.

Such comparison information could be based on a simple time to completion parameter or could involve a more complex comparison based on time to recognize and execute individual steps and weight such individual step performance based on the complexity of the individual steps. Such comparison information provides the opportunity to track and evaluate the performance of the user over time and thereby to monitor for such aspects as: progression of a degenerative condition; unusually rapid or slow development of a skill or aptitude. Such information may be used as an early warning of onset of an illness, or a learning disability, or suitability for a particular career or activity.

Collating the performance of similar activities by one or more groups of individuals and comparing the performance of a user with the average performance, or some other function of the performance, of one or more groups provides the opportunity to calibrate or evaluate the performance of the user being compared. Correlating with choices of others in similarly selected and performing groups provides statistical guidance for career or interest choices and flags possible disorders for further evaluation.

The resulting comparative information and reporting of characteristics of other individuals with similar performance can be used to provide feedback to the user to rate their performance and thereby determine, for example, their susceptibility to a degenerative condition or a learning disability; or suitability for a particular career or activity.

A significant benefit to the user, or to the user's care giver or guardian, would be long-term monitoring of the user's abilities which could provide: early detection of degenerative conditions or of disorders such as dementia, Alzheimer's, hearing impairment, dyslexia, eye disorders, such as glaucoma, macular degeneration, etc., or to provide information regarding aptitude for careers: for music, perfect tone; for mathematics, speed of arithmetic; etc., or to provide information regarding interests, by monitoring choices including, but not limited to, book selection, CD, DVD or BluRay discs, movies, travel locations, educational courses, career choices, choice of crossword areas, etc.

Monitoring the activities of the users provides the opportunity for targeted advertising addressing areas of interest to the user. Potential areas of interest include, but are not limited to: books; CD, DVD, BluRay discs; movies; travel locations; educational courses; medical treatment opportunities.

Such advertising opportunities provide the basis for a business model or financial mechanism to enable a viable business. A business model could also include an annual or monthly user fee for individuals authorized to access and use the platform. The business model could also include links to other web-sites, e.g. sudoku or crossword sites; or medical treatment sites; or educational sites.

### BRIEF DESCRIPTION OF THE DRAWINGS

FIG. 1 is a schematic type illustration of a preferred embodiment of the performance assessment and monitoring system according to the invention.

FIG. 2 Depicts the steps involved in a system for classifying at least one performance parameter of at least one activity by a specific individual according to the invention.

### DETAILED DESCRIPTION OF THE INVENTION

The preferred embodiment of the invention includes a web-site or Internet portal which individuals can use to perform will defined activities or tasks with measurable and quantifiable aspects. At least a portion of the data extracted from the quantifiable aspects is stored in a secure manner as personal data. Current personal data is compared with statistically combined personal data generated from a large number of individual personal data. Current personal data is also compared with similar previously acquired personal data to monitor any change in capabilities of the individual and to assess the significance of such changes, if any.

Activities or tasks include, but are not limited to: crossword or sudoku puzzles, hearing and visual acuity tests, memory tests. Activities or tasks can be performed on personal computers and may involve consumer devices including, but not limited to, audio, video and haptic devices.

The results of such comparisons are used to predict likely future developments of conditions of the individual or aptitude or suitability of the individual for particular life choices.

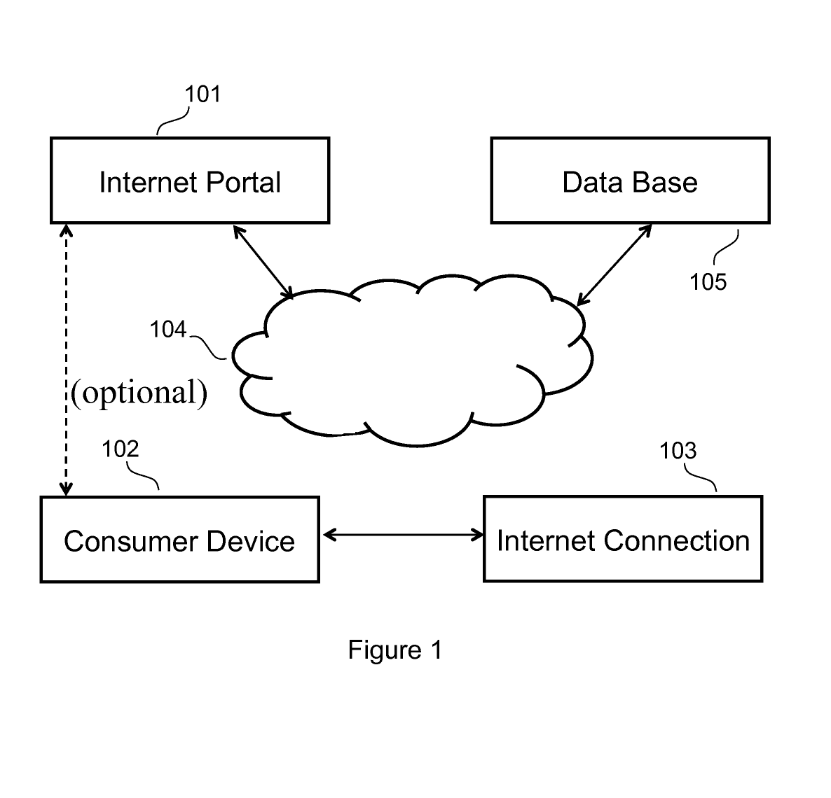

A preferred embodiment of this invention is illustrated in and described with reference to FIG. 1. The preferred embodiment includes a web-site or an internet portal 101 through which activities are analyzed and allocated generalized characteristics by steps such as the following.

(a) Categorizing the activity in terms of a set of relationship to life of individuals to assign the activity a category. Categories include but are not limited to: physical or mental development; health or well being; education related; sport or entertainment; business.

(b) Ranking the activity in terms of significance of such activity to assign the activity a rank. Ranking includes but is not limited to determining the level of difficulty of an activity or determining the level of difficulty of an activity for a specific age group or gender group, or determining the level of difficulty of an activity for individuals with specific experience or training or determining the level of difficulty of an activity for individuals with specific physical or mental capabilities.

(c) Identifying at least one measurable and quantifiable parameter of the activity and analyzing and recording, in terms of one or more measurable and quantifiable parameter, the performance of a large number of individuals in executing the activity so as to assign the activity a value set that reflects an assessment of the performance of the activity.

(d) collating aspects of the resulting category, rank and value set to allocate generalized characteristics to the activity which facilitates automatically classifying the performance of the activity by an individual and comparing or collating the performance of the activity by such an individual with that of a large number of other individuals.

In the preferred embodiment, illustrated in FIG. 1, an individual could access the Internet by means of an consumer device 102. Such consumer devices include but are not limited to: a computer, lap-top or other type; a tablet such as an iPad; an iPhone; an iPod; a cell phone; a TV or other consumer device which has either direct or indirect Internet Connection 103 which provides access via the Internet (or cloud) 104 to the Internet Portal 101 and a Data Base 105 which may include secure storage. For purposes of this invention an Internet portal include any device or software program, such as a web-site that allows connection to the Internet.

The individual uses such a consumer device to perform an activity that has been previously allocated a set of generalized characteristics. Such activities include, but are not limited to: games, such as, sudoku, crosswords, chess, poker, game-boy; learning activities, such as, mathematics; music; language; “digital” activities, such as, typing, texting, dialing phone numbers.

The activity performed by the individual is analyzed and at least one performance parameter of the activity is classified and recorded. Performance parameters provide a measure of competence or skill or dexterity or acuity or knowledge or experience etc. relating to the activity. Examples of a performance parameter could be, (in the case of typing or texting) one of the following: number spelling errors; letter transposition as a marker for dyslexia.

In the case of games (for example sudoku) a performance parameter could be a measurement such as; time to completion, missed easy steps in conjunction with the complexity of the puzzle. Aspects such as types of errors, repeating the same or different errors in periodically repeated versions.

The performance parameters of the activity by the individual are collated with the performance parameters of similar activities by a group of individuals with characteristics similar to those of the individual currently performing the activity. In this manner the performance of the individual can be compared with the performance of a peer group to assess their relative performance.

The performance parameters of the activity by the individual can also be collated with the performance parameters of similar activities previously performed by the same individual to assess their relative performance with respect to historical performance of the same or a similar activity. In this manner the performance of the individual can be tracked over time to monitor, for example, normal development or, as another example, evolution of a deteriorating condition.

The resulting classification of a performance parameter can be further analyzed to assess personal (physical and/or mental) development or to assess aptitude for a particular discipline (study subject, career, etc.) or to assess susceptibility to an illness (malignant condition, etc.) or to assess progress of an illness (malignant condition, etc.). For the purposes of this invention the term illness includes, but is not limited to: malignant condition; degenerative disease; etc. For purposes of this invention the term “aptitude for a discipline” includes, but is not limited to: aptitude for a particular career; aptitude for a particular field of study subject; aptitude for a particular hobby; etc.).

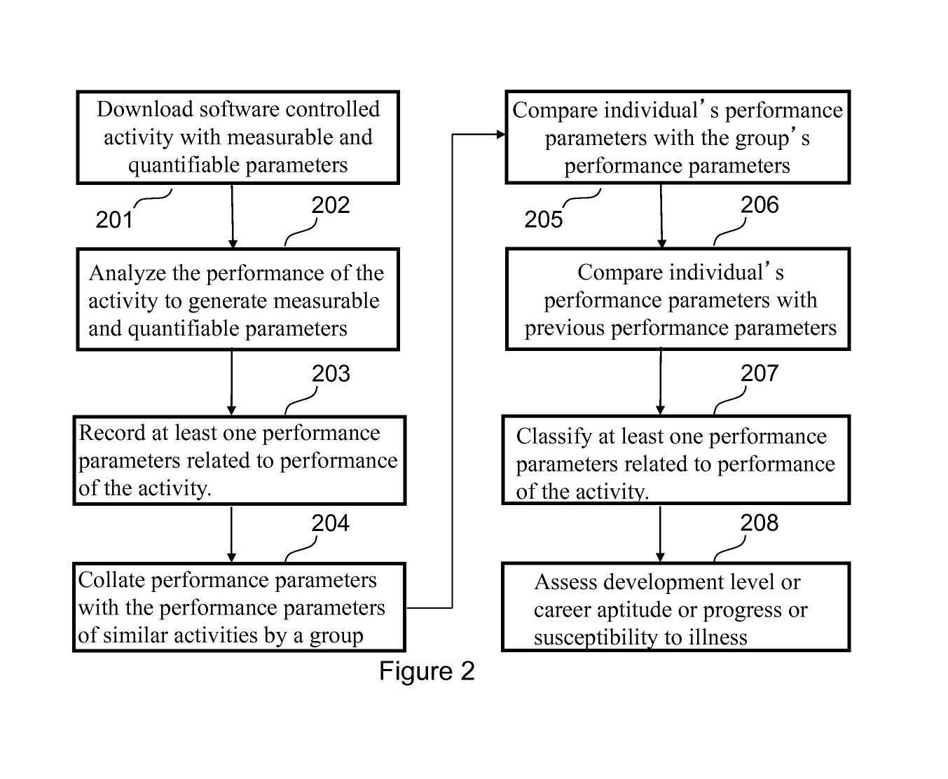

A specific example of monitoring and tracking an activity is described and illustrated with respect to FIG. 2 where hearing acuity is monitored and tracked or aptitude for a musical hobby or career is assessed.

In this example an audio based game is downloaded from a web-site or interne portal by an individual. An aspect of the game involves identifying tones or tone sequences. Playing the game constitutes selecting an activity with one or more measurable and quantifiable aspects. In this example measurable and quantifiable aspects could include, but not be limited to, the ability to identify changes in tones or tone sequences with quantifiable aspects related to the magnitude or duration of tone changes that are successfully detected by the individual or the speed with which such changes are detected by the individual.

The performance of the individual playing the game is analyzed in terms of one or more of the quantifiable aspects which constitute performance parameters. One or more of these performance parameters may be recorded for future reference and may also be correlated with the performance parameters others by collating the performance of the specific individual with the average performance parameters of a group performing the same activity or activities with similar characteristics.

At least one performance parameter of the activity (playing the game) as performed by the specific individual may classified by comparing the individual's performance parameters with that of the group and optionally with previous performance parameters of the same or a similar activity previously performed by the individual and stored via the Internet on a secure storage device.

The resulting classification of one or more performance parameters may be further analyzed to assess personal development. In the present example, for the case of the individual being a child, such personal development could be to assess if the individual had age appropriate hearing acuity.

The resulting classification of one or more performance parameters may be further analyzed to assess the individual's aptitude for a particular discipline. In the present example such a particular discipline could be a career or hobby as a musician (for an individual with a particularly high performance parameter (higher values indicting better performance).

The resulting classification of one or more performance parameters may be further analyzed to assess the susceptibility to, or progress of, an illness or malignant condition. In the present example, for the case of the individual with previously recorded similar performance parameters, such an illness or malignant condition could be a degradation of hearing acuity.

The above example is an embodiment that could use one or more speakers to monitor and track hearing acuity or musical aptitude and could involve a microphone to detect user generated sounds. Many other examples of embodiments will be apparent to persons skilled in the art and are included in the invention. For example games that require distinguishing colors to monitor and track color vision could be activities with measurable and quantifiable parameters. Downloadable software programs that control other consumer devices, such as bio-sign monitors, to monitor and track physiological attributes could be activities with measurable and quantifiable parameters.

The preferred embodiment is illustrated in and described with respect to FIG. 2, where the steps involved in a system for classifying at least one performance parameter of at least one activity by a specific individual are depicted.

Step 201 involves downloading a selected activity that has one or more measurable and quantifiable aspects using a platform, such as a web-site or Internet portal.

Step 202 involves analyzing the performance of the activity to generate one or more measurable and quantifiable aspect that can be represented as one or more measurable and quantifiable parameters.

Step 203 involves recording at least one of the performance parameters related to the performance of the activity.

Step 204 involves collating the performance parameters of the activities with the performance parameters of the activities by a group of individuals with similar characteristics.

Step 205 involves comparing the performance parameters of the specific individual with the average performance parameters of the group.

Step 206 involves comparing the performance parameters of the specific individual with previous performance parameters of similar activity by the specific individual.

In Step 207 at least one performance parameter of the activity by said specific individual is classified in order to more readily rank the performance of the individual with the average performance of a group of individuals with similar characteristics to the individual performing the activity.

Step 208 involves using the resulting performance parameter in combination with performance parameters of others and previous performance parameters of the individual to assess such aspects as: physical or mental development level; or aptitude for a hobby, field of study or career; or progress of or susceptibility to an illness or malignant condition.

The invention also includes a method and system for allocating generalized characteristics to an activity in order to facilitate comparing and ranking the performance parameters of disparate activities. Such a method and system includes categorizing a selected activity in terms of a set of relationships to the life of an individual in order to assign the activity a category. It further includes ranking the selected activity in terms of significance or impact of such activity.

It further includes identifying at least one measurable and quantifiable parameter associated with the activity. It also includes analyzing and recording, in terms of such measurable and quantifiable parameters, the performance of a large number of individuals in executing the selected activity in order to assign the activity a value set.

The invention further includes collating the category, rank and value set thereby allocating generalized characteristics to the activity. The resulting generalized characteristics enable comparing performance parameters of different activities that in turn enables a more comprehensive ranking of an individual's performance over a broader range of activities.

For example, allocating generalized characteristics to different games would enable the performance parameters on an individual playing one game to be compared and ranked with the performance parameters of groups of individuals playing different games.

As another example, allocating generalized characteristics to different activities, such as: spelling accuracy during the activity of typing; speed and accuracy of the activity of dialing phone numbers; or incidents of incorrect password entry when accessing e-mail; would enable the performance parameters on an individual performing any one of these activities to be compared and ranked with the performance parameters of groups of individuals performing the other activities.

In this (above) example, such the performance parameters of disparate activities could be used to monitor for progressive degenerative conditions such as Alzheimer's disease. In such cases ranking information would provide information on the emergence or progression of the disease. Such information could be automatically reported to the individual's physician or other authorized care giver.

The above embodiments and examples are intended to be illustrative and not limiting. Many other examples of embodiments will be apparent to persons skilled in the art and are included in the invention. The scope of this invention should be determined with reference to the specification, the drawings, the appended claims, along with the full scope of equivalents as applied thereto.

What is claimed is:

1. A system for classifying at least one performance parameter of at least one activity by a specific individual, said system comprising:
a platform that provides activities, said activities having at least one measurable and quantifiable aspects;
analyzing the performance of said activities;
recording performance parameters related to the performance of said activities;
collating the performance parameters of said activities with the performance parameters of said activities by a group of individuals with similar characteristics;
comparing the performance parameters of said specific individual with the average performance parameters of the group;
whereby at least one performance parameter of said activity by said specific individual is classified.

2. The system of claim 1, additionally comprising comparing the performance parameters of said specific individual with previous performance parameters of similar activity by said specific individual,

3. The system of claim 1, wherein the classification of a performance parameter is further analyzed to assess personal physical development.

4. The system of claim 1, wherein the classification of a performance parameter is further analyzed to assess personal mental development.

5. The system of claim 1, wherein the classification of a performance parameter is further analyzed to assess aptitude for a discipline.

6. The system of claim 1, wherein the classification of a performance parameter is further analyzed to assess susceptibility to an illness.

7. The system of claim 1, wherein the classification of a performance parameter is further analyzed to assess progress of an illness.

8. The system of claim 1, wherein the system includes an Internet portal.

9. The system of claim 1, wherein said system includes a device, selected from one or more of the following: a computer; an iPhone; an iPod; an iPad; a cell phone; or other Internet accessing devices.

10. The system of claim 1, wherein said system includes a secure storage device.

11. The system of claim 1, wherein said activities include activities selected from one or more of the following: games; learning activities; “digital” activities;

12. The system of claim 11, wherein games include games selected from one or more of the following: sudoku; crosswords; chess; poker; game-boy.

13. The system of claim 11, wherein learning activities include learning activities selected from one or more of the following: mathematics; music; language.

14. The system of claim 11, wherein digital activities include digital activities selected from one or more of the following: typing; texting; dialing phone numbers.

15. The system of claim 1, wherein a performance parameter is selected from one or more of the following: number spelling errors; letter transposition for dyslexia; types of errors; periodically repeated versions of errors.

16. The system of claim 1, wherein a performance is a measurement selected from one or more of the following: time to completion/complexity; types of errors; missed easy steps; periodically repeated versions.

17. The system of claim 1, wherein generalized characteristics are allocated to the activity.

18. A computer program product for classifying at least one performance parameter of at least one activity by a specific individual, comprising: one or more machine readable storage media; programming logic provided by said machine readable media for downloading a program to: control activities that have one or more measurable and quantifiable aspects; analyze the performance of said activities; record performance parameters related to the performance of said activities; collate the performance parameters of said activities with the performance parameters of said activities by a group of individuals with similar characteristics; compare the performance parameters of said specific individual with the average performance parameters of the group; whereby at least one performance parameter of said activity by said specific individual is classified.

19. The computer program product of claim 18, further comprising a program to compare the performance parameters of said specific individual with previous performance parameters of similar activity by said specific individual.

20. A system for allocating generalized characteristics to an activity, said system comprising:
categorizing said activity in terms of a set of relationships to life of an individual to assign the activity a category;
ranking said activity in terms of significance of such activity to assign the activity a rank;
identifying at least one measurable and quantifiable parameter of said activity;
analyzing and recording, in terms of said measurable and quantifiable parameter, the performance of a large number of individuals in executing said activity to assign the activity a value set;
and collating the category, rank and value set thereby allocating generalized characteristics to the activity.
